(19) United States
(12) Patent Application Publication
(10) Pub. No.: US 2020/0152336 A1
(43) Pub. Date: May 14, 2020

(54) AUTOMATED PERSONALIZED ANNOTATION OF CLINICAL GUIDELINES

(71) Applicant: International Business Machines Corporation, Armonk, NY (US)

(72) Inventors: Samuel Osebe, Nairobi (KE); Charles Muchiri Wachira, Nairobi (KE); Sekou Lionel Remy, Nairobi (KE); John Mbari Wamburu, Nairobi (KE); Katherine Anne Lyon Tryon, Nairobi (KE); Aisha Walcott, Nairobi (KE)

(21) Appl. No.: 16/186,506

(22) Filed: Nov. 10, 2018

(51) Int. Cl. G16H 70/20; G06F 17/24; G06F 17/22; G06F 17/27

(52) U.S. Cl. CPC G16H 70/20; G06F 17/241; G06F 17/2705; G06F 17/277; G06F 17/2276

(57) ABSTRACT

A guideline annotation method, system, and computer program product, include extracting a medical data point from a medical guideline, determining an attribute context of the medical data point, finding a related literature to the medical guideline, determining an expert practice as a difference between the medical guideline and an expert operating procedure, and annotating and outputting a new medical guideline including the related literature and the expert practice.

### BACKGROUND

The present invention relates generally to a guideline annotation method, and more particularly, but not by way of limitation, to a system, method, and recording medium for using natural language understanding to extract attributes, their related measurements, decision points and actions from guideline narratives.

Several organizations have developed clinical decision support tools for both patients and healthcare providers, which range from general decision support to more disease-specific decision support.

Conventionally, there is a limited ability to easily adapt these tools for different environments such as different geographies or different specialties, as each healthcare system has its own rules focused on the responsibilities and powers vested in different specialties within different geographies, local guidelines, and agreements by panels. For example, a specific demographic may exhibit side effects from a treatment plan, whereas a different demographic may benefit immensely from the same treatment plan.

However, conventionally the process of guideline ingestion remains a mainly manual process, requiring specialized individuals to break down the information contained in the guidelines and put them into the system. Also, there is a steep knowledge curve associated with learning guideline ontologies as they have complex syntax. This further complicates the process of ingestion.

Guidelines are expressed in multiple ways including narratives, diagrams, and sequence of steps or standard operating procedures. But, there is a problem in the art that the fact that the process of guideline ingestion is tedious and slow means that it is difficult to update guidelines and/or deviate from the guidelines without much additional work.

### SUMMARY

In an exemplary embodiment, the present invention can provide a computer-implemented guideline annotation method, the method including extracting a medical data point from a medical guideline, determining an attribute context of the medical data point, finding a related literature to the medical guideline, determining an expert practice as a difference between the medical guideline and an expert operating procedure, and a annotating and outputting a new medical guideline including the related literature and the expert practice.

One or more other exemplary embodiments include a computer program product and a system.

Other details and embodiments of the invention will be described below, so that the present contribution to the art can be better appreciated. Nonetheless, the invention is not limited in its application to such details, phraseology, terminology, illustrations and/or arrangements set forth in the description or shown in the drawings. Rather, the invention is capable of embodiments in addition to those described and of being practiced and carried out in various ways and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

Aspects of the invention will be better understood from the following detailed description of the exemplary embodiments of the invention with reference to the drawings, in which

FIG. 1 exemplarily shows a high-level flow chart for a guideline annotation method 100;

FIG. 2 exemplarily depicts an implementation of step 101 of the method 100 according to an embodiment of the present invention;

FIG. 3 exemplarily depicts an implementation of step 102 of the method 100 according to an embodiment of the present invention;

FIG. 4 exemplarily depicts an implementation of step 103 of the method 100 according to an embodiment of the present invention;

FIG. 5 exemplarily depicts an implementation of step 104 of the method 100 according to an embodiment of the present invention;

FIG. 6 exemplarily depicts an implementation of step 105 of the method 100 according to an embodiment of the present invention;

FIG. 7 exemplarily depicts an annotated guideline according to step 101 of the method 100 according to an embodiment of the present invention;

FIG. 8 exemplarily depicts a system implementation of the method 100 according to an embodiment of the present invention;

FIG. 9 depicts a cloud computing node 10 according to an embodiment of the present invention;

FIG. 10 depicts a cloud computing environment 50 according to an embodiment of the present invention; and

FIG. 11 depicts abstraction model layers according to an embodiment of the present invention.

### DETAILED DESCRIPTION

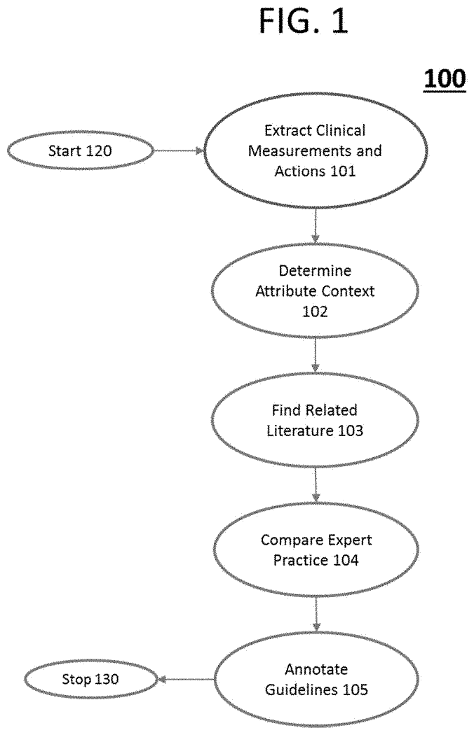

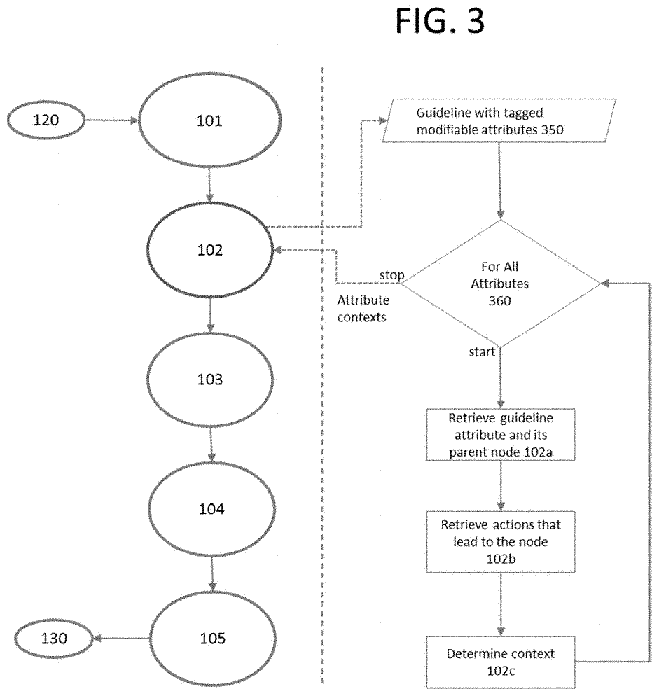

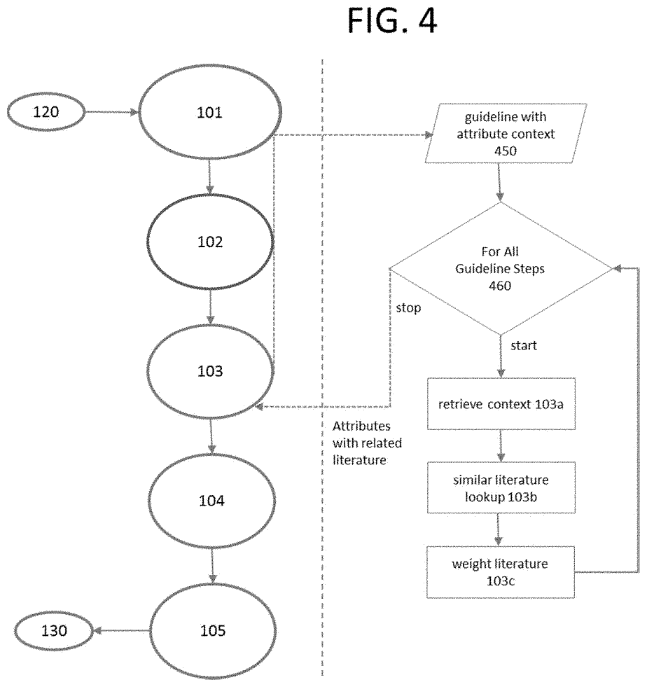

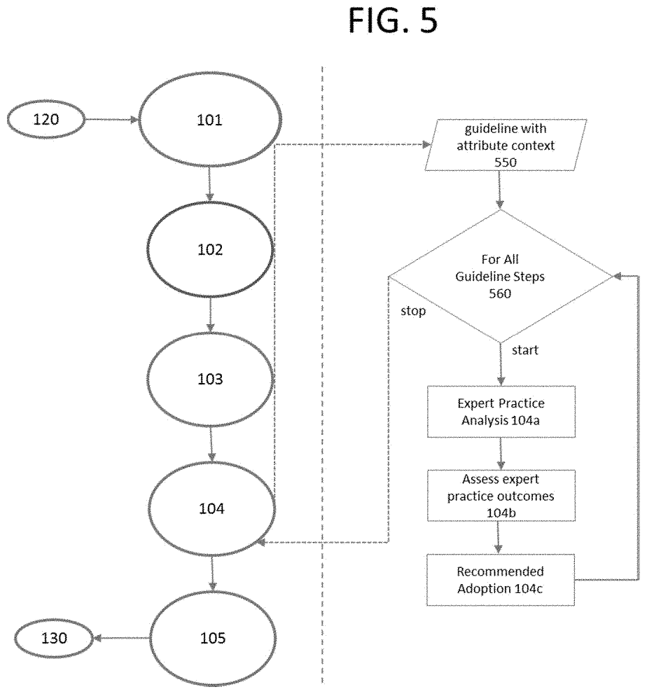

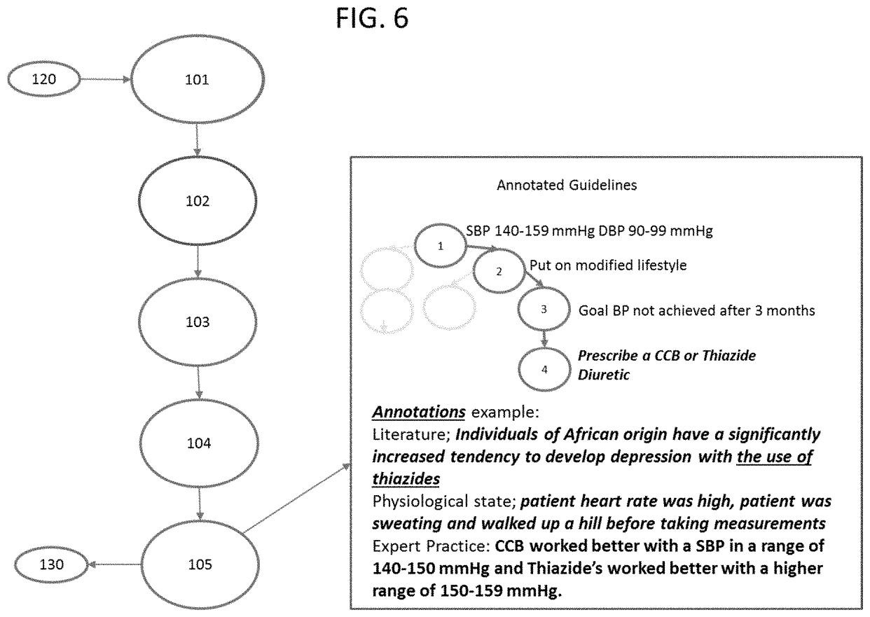

The invention will now be described with reference to FIG. 1-11, in which like reference numerals refer to like parts throughout. It is emphasized that, according to common practice, the various features of the drawing are not necessarily to scale. On the contrary, the dimensions of the various features can be arbitrarily expanded or reduced for clarity.

With reference now to the example depicted in FIG. 1, the guideline annotation method 100 includes various steps to use Natural Language Understanding (NLU) to determine guideline attributes and resulting actions from guideline narratives, which in turn make up guideline nodes. These nodes are then analyzed for chronology and structured into a model that is understandable by a computer while still maintaining the semantics of the information contained in the guideline. This information model is stored and made accessible, for example, via querying and retrieval mechanism.

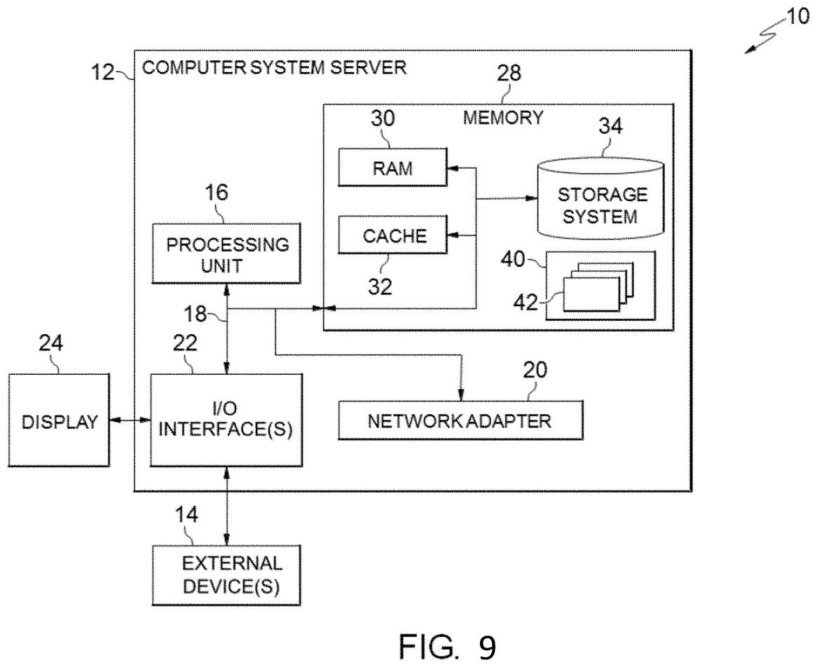

As shown in at least FIG. 9, one or more computers of a computer system 12 according to an embodiment of the present invention can include a memory 28 having instructions stored in a storage system to perform the steps of FIG. 1.

The guideline annotation method 100 according to an embodiment of the present invention may act in a more sophisticated, useful and cognitive manner, giving the impression of cognitive mental abilities and processes related to knowledge, attention, memory, judgment and evaluation, reasoning, and advanced computation. A system can be said to be “cognitive” if it possesses macro-scale properties—perception, goal-oriented behavior, learning/memory and action—that characterize systems (i.e., humans) generally recognized as cognitive.

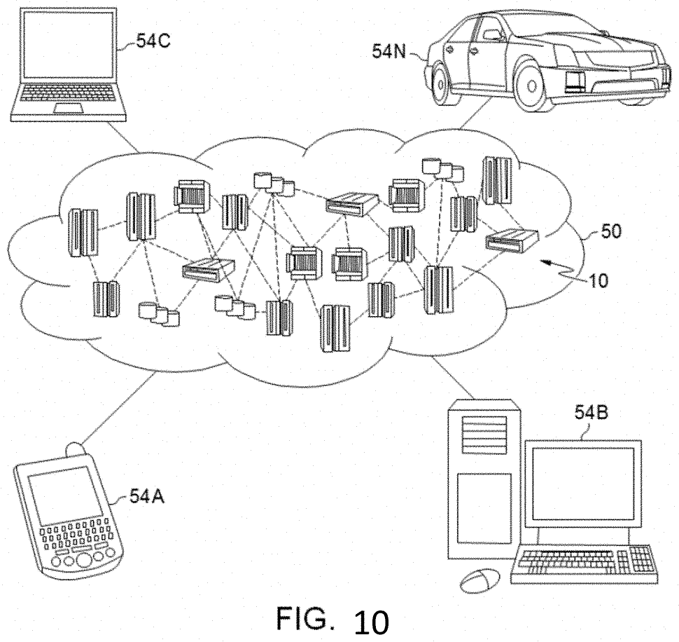

Although one or more embodiments (see e.g., FIGS. 9-11) may be implemented in a cloud environment 50 (see e,g., FIG. 10), it is nonetheless understood that the present invention can be implemented outside of the cloud environment.

With reference generally to FIGS. 1-8, the invention uses NLU to extract attributes, their related measurements, decision points and actions from guideline narratives. Attributes include clinical indicators (e.g., pulse, blood pressure, etc.) while measurements would be the thresholds that guidelines set as a decision criteria for actions. Attributes can be extracted based on a corpus of clinical indicators while NLU association techniques would then be used to determine which measurement numbers are associated with which attributes. This is followed by determining relationships between attributes and actions and binding this into a data structure to form nodes for the larger data structure. Given the nodes, the next step is determining the chronological relations between the nodes by examining the use of conjunctions in the narrative. This is then packed into a tree structure whose root node would be the first step of the guideline and the leaf nodes would be the final action taken by a caregiver.

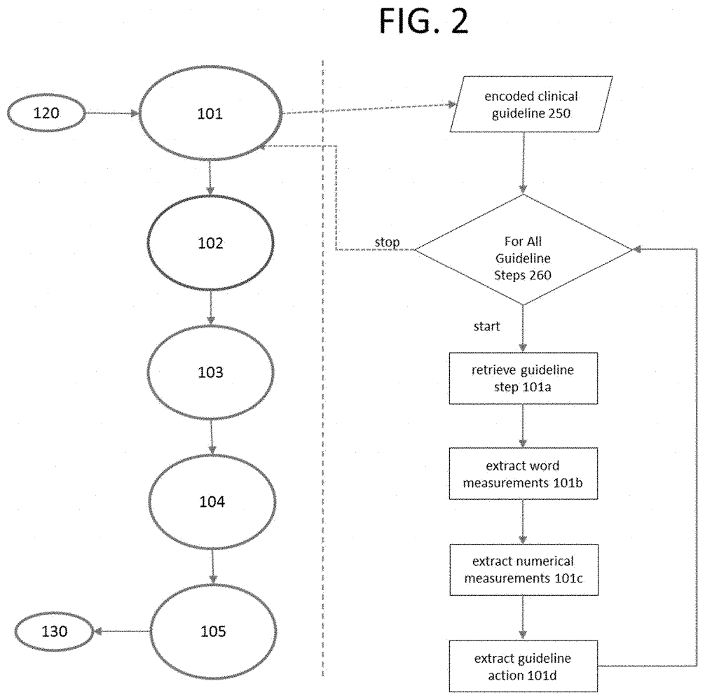

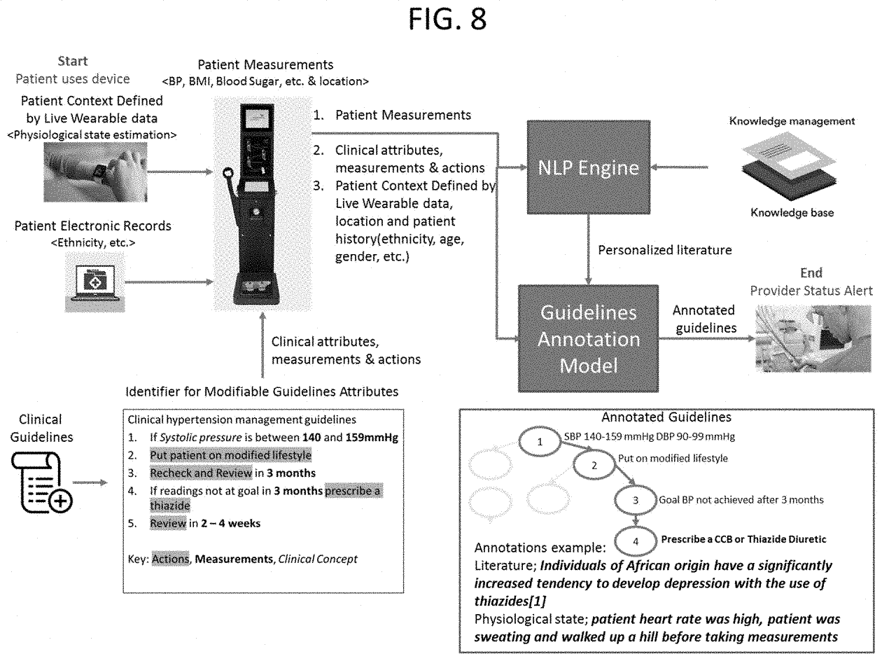

Referring to FIGS. 1 and 8, in step 101, clinical measurements and actions are extracted. That is, as shown in FIGS. 2 and 8, clinical guidelines 250 are encoded and ingested. For each of the guidelines ingested, the subroutine 260 is performed in a loop to retrieve guidelines in step 101a, extract word measurements in step 101b, extract numerical measurements in step 101c, and extract guidelines actions in step 101d. To extract word measurements, the method may use an NLP parse tree to determine adjectives and adverbs which are indexed as the node and the location of the attribute. To extract numerical measurements, the method may use NLU to determine numbers that can be categorized as sensor readings (e.g., 80 bpm for pulse) and then index the node and the location of the attribute. Step 101d extract actions by using NLU, medical ontology and concept dictionary to identify guideline actions. An action is modifiable if it contains a number or an adjective related to it (e.g., perform procedure five times). The actions are indexed with the node and the location of the attribute.

Moreover, in step 101 patient measurements are extracted and the patient context can be defined by wearable data, location, patient history, etc.

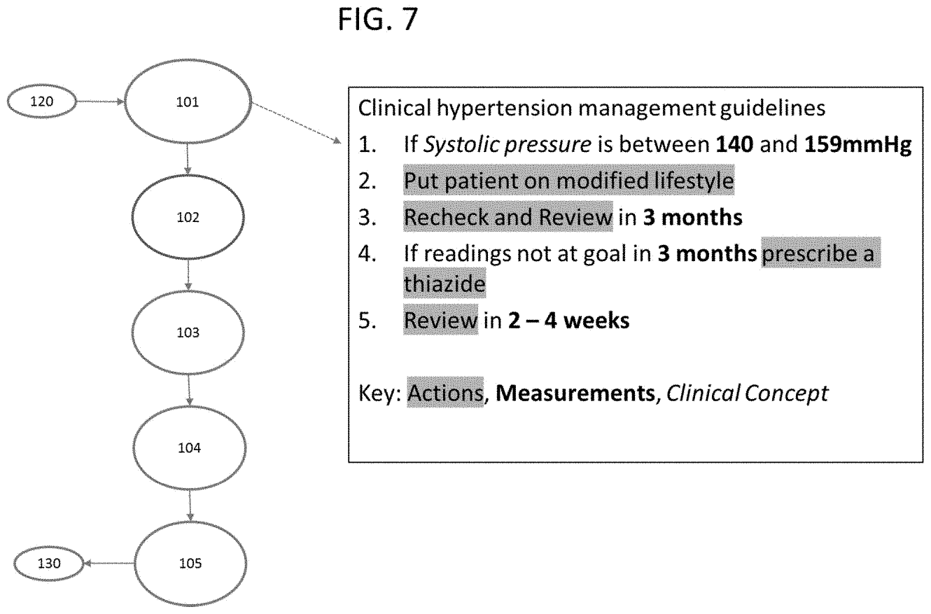

For example, FIG. 7 depicts an exemplary result of step 101 in which actions, measurements, and clinical concepts are extracted from an exemplary clinical hypertension management guideline. The highlighted words embody actions, the bold words embody measurements, and the italicized words embody a clinical concept.

With reference to FIGS. 1, 3, and 8, step 102 determines the context of the attributes extracted from step 101. In step 102, the guidelines are tagged with modifiable attributes 350 in a subsequence where for all attributes, guideline attributes and its parent node are retrieved in step 102a, actions that lead to the node are retrieved in step 102b, and the context is determined in step 102c. More specifically, the guideline description are retrieved from the guideline data repository. This acts as a parameter to the context equation. And, given an attribute in a node, traverse the guideline from the root node to retrieve a list of actions leading to the attribute. The closer the action is to the attribute, the more it is weighted. For example, “Weight;=(number of levels)ᵢ/sum(weights)”. This serves as the second parameter. In step 102c, the context is determined by “Context=(guideline description)+[weighted(actions)]+patient-setting”.

With reference to FIGS. 1, 4, and 8, in step 103, related literature is found for annotating the guidelines. More specifically, the attribute context 450 is used to retrieve context in step 103a, lookup similar literature in step 103b, and weight the value of the literature in step 103c. That is, steps 103a-103c fetch similar literature by, for each modifiable node, retrieving the node's context, searching literature abstracts and conclusions using the context as search parameters, and returning list of literatures. Then, the literature is weighted and ranked based on context. This should be affected by the weighted action list. The rank can be determined by, for example, “Rank=f1 (guideline description)+f2(w1(actions) . . . wn(actions))”. Each node is tagged with a ranked literature. The related literature is ranked based on an overlap of the attribute context and a computer summary of the related literature.

With reference to FIGS. 1, 5, and 8, in step 104, the guidelines are compared with expert practice. More specifically, in steps 104a-104c, the expert practice in the guideline context is analyzed, the outcomes of the expert practice are assessed (e.g., results of practice), and an adoption of the practice is recommended if helpful. For example, the assessment is conducted by cross referencing the expert practice with success rates from medical journals. That is, steps 104a-104c determine differences between guidelines and expert operating procedures. Then, it is determined if expert deviations from guidelines are truly beneficial before adopting them.

With reference to FIGS. 1, 6, and 8, in step 105, the guidelines are annotated to give a provider additional information to make decisions in the guidelines. For example, the ingested guideliens of FIG. 7 are annotated to show that there are four steps to treat the condition. In step 4, a CCB or a thiazide can be prescribed. The guidelines are annotated with examples from literature or expert practice to show that individuals of African origin have a significantly increased tendency to develop depression with the use of thiazides. Therefore, the doctor can decide to prescribe a CCB instead of a thiazide if the patient is of African origin. Also, additional context can be provided on the guidelines to show the state of the patient, as for example, “patient heart rate was high, patient was sweating and walked up a hill before taking measurements”. Thus, the doctor an take another measurement at a later time once the patient's heart rate comes down. And, expert comparison practice can be included on the guideline to further assist a doctor in deciding treatment (e.g., “CCB worked better with a SBP in a range of 140-150 mmHg and thiazide's worked better with a higher range of 150-159 mmHg”).

Therefore, the guidelines which merely state to prescribe a CCB or thiazide with no additional guidelines are annotated to include context about the patient from attributes of the patient, literature, expert practice, etc.

Thus, the method 100 can provide a technique for storing guideline information as a knowledge graph to increase the ability of systems to autonomously share information by providing a uniform representation of digital guidelines. The use of NLU to determine attributes, actions and their relationships enables compatibility for cognition of computers. Using NLU to determine actions and their relationships improves current techniques of knowledge acquisition and structuring from free text. This presents a platform on which other innovations can build on, reducing time to market for other systems. Also, a healthcare provider is provided with additional information without manual search to better deciding treatment plans for a patient at a lower cost.

Exemplary Aspects, Using a Cloud Computing Environment

Although this detailed description includes an exemplary embodiment of the present invention in a cloud computing environment, it is to be understood that implementation of the teachings recited herein are not limited to such a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client circuits through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

Referring now to FIG. 9, a schematic of an example of a cloud computing node is shown. Cloud computing node 10 is only one example of a suitable node and is not intended to suggest any limitation as to the scope of use or functionality of embodiments of the invention described herein. Regardless, cloud computing node 10 is capable of being implemented and/or performing any of the functionality set forth herein.

Although cloud computing node 10 is depicted as a computer system/server 12, it is understood to be operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop circuits, multiprocessor systems, microprocessor-based systems, set top boxes, programmable consumer electronics, network PCs, minicomputer systems, mainframe computer systems, and distributed cloud computing environments that include any of the above systems or circuits, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing circuits that are linked through a communications network. In a distributed cloud computing environment, program modules may be located in both local and remote computer system storage media including memory storage circuits.

Referring again to FIG. 9, computer system/server 12 is shown in the form of a general-purpose computing circuit. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external circuits 14 such as a keyboard, a pointing circuit, a display 24, etc.; one or more circuits that enable a user to interact with computer system/server 12; and/or any circuits (e.g., network card, modem, etc.) that enable computer system/server 12 to communicate with one or more other computing circuits. Such communication can occur via Input/Output (I/O) interfaces 22. Still yet, computer system/server 12 can communicate with one or more networks such as a local area network (LAN), a general wide area network. (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, circuit drivers, redundant processing units, external disk drive arrays, RAID systems, tape drives, and data archival storage systems, etc.

Referring now to FIG. 10, illustrative cloud computing environment 50 is depicted. As shown, cloud computing environment 50 comprises one or more cloud computing nodes 10 with which local computing circuits used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing circuit. It is understood that the types of computing circuits 54A-N shown in FIG. 10 are intended to be illustrative only and that computing nodes 10 and cloud computing environment 50 can communicate with any type of computerized circuit over any type of network and/or network addressable connection (e.g., using a web browser).

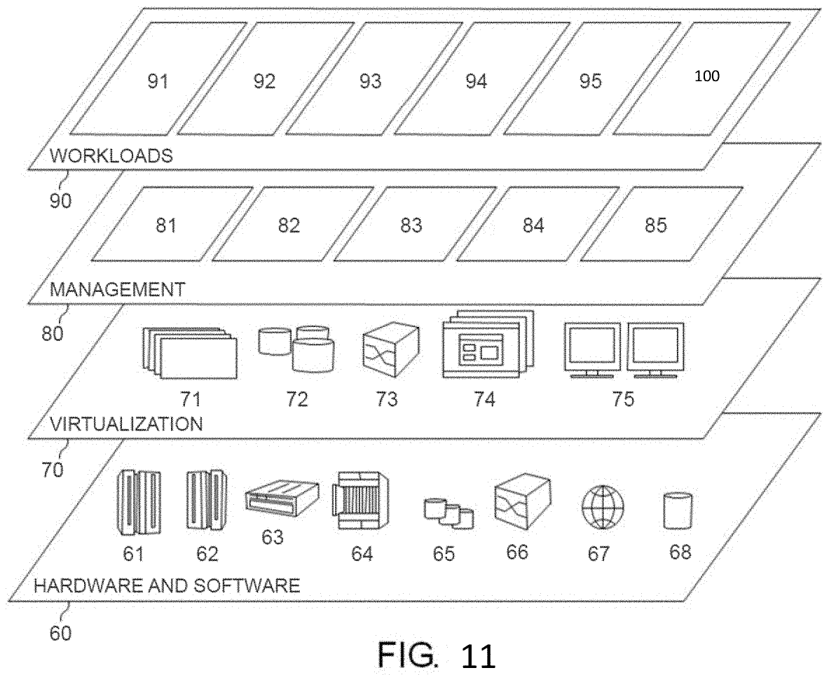

Referring now to FIG. 11, an exemplary set of functional abstraction layers provided by cloud computing environment 50 (FIG. 10) is shown. It should be understood in advance that the components, layers, and functions shown in FIG. 11 are intended to be illustrative only and embodiments of the invention are not limited thereto. As depicted, the following layers and corresponding functions are provided:

Hardware and software layer 60 includes hardware and software components. Examples of hardware components include; mainframes 61; RISC (Reduced Instruction Set Computer) architecture based servers 62; servers 63; blade servers 64; storage circuits 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual. networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and, more particularly relative to the present invention, the guideline annotation method 100.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device:, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” progamming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over—technologies found in the marketplace, or to enable others of ordinary skill in the art to understand the embodiments disclosed herein.

Further, Applicant's intent is to encompass the equivalents of all claim elements, and no amendment to any claim of the present application should be construed as a disclaimer of any interest in or right to an equivalent of any element or feature of the amended claim.

What is claimed is:

1. A computer-implemented guideline annotation method, the method comprising:
extracting a medical data point from a medical guideline;
determining an attribute context of the medical data point;
finding a related literature to the medical guideline;
determining an expert practice as a difference between the medical guideline and an expert operating procedure; and
annotating and outputting a new medical guideline including the related literature and the expert practice.

2. The computer-implemented method of claim 1, wherein the medical data point comprises:
a clinical concept;
a measurement; and
an action.

3. The computer-implemented method of claim 2, wherein the extracting extracts the clinical concept, the measurement, and the action by:
extracting word measurements to identify the clinical concept using a parse tree to determine at least one of adjectives and adverbs in the medical guideline and indexing a node and a location of the at least one of adjectives and the adverbs in the medical guideline;
extracting numerical measurements to determine numbers in the medical guideline as the measurement and indexing a node and a location of the numbers in the medical guideline; and
extracting the action using a Natural Language Understanding (NLU) of a medical ontology and a concept dictionary to identify the action and indexing a node and a location of the action.

4. The computer-implemented method of claim 1, wherein the determining the attribute context includes:
retrieving a list of actions leading to an attribute in the medical data point; and
given the attribute in a node of the medical guideline, traversing the medical guideline from a root node,
wherein the attribute context is weighted as more accurate based on a closeness of the action to the attribute.

5. The computer-implemented method of claim 4, wherein the finding the related literature uses the attribute context as a search parameter for finding relevant literature, and
wherein the related literature is ranked based on an overlap of the attribute context and a computer summary of the related literature.

6. The computer-implemented method of claim 1, further comprising:
receiving a clinical measurement of a patient; and
additionally annotating and outputting the new medical guideline with the clinical measurement of the patient.

7. The computer-implemented method of claim 1, embodied in a cloud-computing environment.

8. A computer program product, the computer program product comprising a computer-readable storage medium having program instructions embodied therewith, the program instructions executable by a computer to cause the computer to perform:
extracting a medical data point from a medical guideline;
determining an attribute context of the medical data point;
finding a related literature to the medical guideline;
determining an expert practice as a difference between the medical guideline and an expert operating procedure; and
annotating and outputting a new medical guideline including the related literature and the expert practice.

9. The computer program product of claim 8, wherein the medical data point comprises:
a clinical concept;
a measurement; and
an action.

10. The computer program product of claim 9, wherein the extracting extracts the clinical concept, the measurement, and the action by:
extracting word measurements to identify the clinical concept using a parse tree to determine at least one of adjectives and adverbs in the medical guideline and indexing a node and a location of the at least one of adjectives and the adverbs in the medical guideline;
extracting numerical measurements to determine numbers in the medical guideline as the measurement and indexing a node and a location of the numbers in the medical guideline; and
extracting the action using a Natural Language Understanding (NLU) of a medical ontology and a concept dictionary to identify the action and indexing a node and a location of the action.

11. The computer program product of claim 8, wherein the determining the attribute context includes:
retrieving a list of actions leading to an attribute in the medical data point; and
given the attribute in a node of the medical guideline, traversing the medical guideline from a root node,
wherein the attribute context is weighted as more accurate based on a closeness of the action to the attribute.

12. The computer program product of claim 11, wherein the finding the related literature uses the attribute context as a search parameter for finding relevant literature, and
wherein the related literature is ranked based on an overlap of the attribute context and a computer summary of the related literature.

13. The computer program product of claim 8, further comprising:
receiving a clinical measurement of a patient; and
additionally annotating and outputting the new medical guideline with the clinical measurement of the patient.

14. A guideline annotation system, said system comprising:
a processor; and
a memory, the memory storing instructions to cause the processor to perform:
extracting a medical data point from a medical guideline;
determining an attribute context of the medical data point;
finding a related literature to the medical guideline;
determining an expert practice as a difference between the medical guideline and an expert operating procedure; and
annotating and outputting a new medical guideline including the related literature and the expert practice.

15. The system of claim 14, wherein the medical data point comprises:
a clinical concept;
a measurement; and
an action.

16. The system of claim 15, wherein the extracting extracts the clinical concept, the measurement, and the action by:
extracting word measurements to identify the clinical concept using a parse tree to determine at least one of adjectives and adverbs in the medical guideline and indexing a node and a location of the at least one of adjectives and the adverbs in the medical guideline;
extracting numerical measurements to determine numbers in the medical guideline as the measurement and indexing a node and a location of the numbers in the medical guideline; and
extracting the action using a Natural Language Understanding (NLU) of a medical ontology and a concept dictionary to identify the action and indexing a node and a location of the action.

17. The system of claim 14, wherein the determining the attribute context includes:
retrieving a list of actions leading to an attribute in the medical data point; and
given the attribute in a node of the medical guideline, traversing the medical guideline from a root node,
wherein the attribute context is weighted as more accurate based on a closeness of the action to the attribute.

18. The system of claim 17, wherein the finding the related literature uses the attribute context as a search parameter for finding relevant literature, and
wherein the related literature is ranked based on an overlap of the attribute context and a computer summary of the related literature.

19. The system of claim 14, further comprising:
receiving a clinical measurement of a patient; and
additionally annotating and outputting the new medical guideline with the clinical measurement of the patient.

20. The system of claim 14, embodied in a cloud-computing environment.
